(19) United States
(12) Patent Application Publication
(10) Pub. No.: US 2002/0192827 A1
(43) Pub. Date: Dec. 19, 2002

(54) Novel vector for C. glabrata and the use thereof

(76) Inventors: Guillaume Cottarel, Arlington, MA (US); Debra Aker Willins, Watertown, MA (US)

Correspondence Address: BURNS DOANE SWECKER & MATHIS L L P, POST OFFICE BOX 1404, ALEXANDRIA, VA, 22313-1404, US

(21) Appl. No.: 10/079,818

(22) Filed: Feb. 22, 2002

Related U.S. Application Data
(63) Continuation-in-part of application No. 09/711,940, filed on Nov. 15, 2000.

(51) Int. Cl. C12N 1/18; C12N 15/74

(52) U.S. Cl. 435/484; 435/320.1; 435/254.22

(57) ABSTRACT

The present invention is directed to a vector for expressing a gene of interest in a Candida species. The vector of the present invention comprises promoter and terminator sequences from a native Candida gene. This vector can be used to complement deleted genes in a Candida species. The present invention is also directed to a method for identifying genes essential for growth of a Candida species. Additionally, the present invention is directed to a method for screening for anti-fungal compounds effective against Candida species.

This application is a continuation-in-part of U.S. application Ser. No. 09/711,940, filed Nov. 15, 2000, the entire content of which is hereby incorporated by reference.

### FIELD OF THE INVENTION

The present invention is directed to novel Candida vectors which can be used to express genes in Candida species. The present invention also is directed to a method of deleting a gene from a Candida species and complementing that gene using the novel Candida vector to express the deleted gene. The present invention further is directed to a method for determining what genes in a Candida species are necessary for growth. Finally, the present invention is directed to a method of screening for anti-fungal compounds.

### BACKGROUND OF THE INVENTION

Fungal infections of humans range from superficial conditions, usually caused by dermatophytes or Candida species, that affect the skin (such as dermatophytoses) to deeply invasive and often lethal infections (such as candidiasis). Pathogenic fungi occur worldwide, although particular species may predominate in certain geographic areas.

In the past 20 years, fungal infections have increased dramatically—along with the numbers of potentially invasive species. Indeed, fungal infections once dismissed as a nuisance have begun to spread so widely that they are becoming a major concern in hospitals and health departments. Fungal infections occur more frequently in people whose immune systems are suppressed (because of organ transplantation, cancer chemotherapy, or human immunodeficiency virus infections), who have been treated with broad-spectrum antibacterial agents, or who have been subject to invasive or surgical procedures (catheters and prosthetic devices, for example). Fungal infections are now important causes of morbidity and mortality of hospitalized patients: the frequency of invasive candidiasis has increased tenfold to become the fourth most common blood culture isolate (Pannuti et al., Cancer 69:2653 (1992)). Many opportunistic fungal infections cannot be diagnosed by usual blood culture and must be treated empirically in severely immunocompromised patients (Walsh et al., Rev. Infect. Dis. 13:496 (1991)).

The Candida genus of yeast are a major cause of life-threatening mucosal and systemic human fungal infections. Species within the Candida genus are so virulent, because they are able to grow in different morphological forms and at elevated temperatures; they are able to switch between different colony or cellular phenotypes; they are able to adhere to host tissue; and they are able to produce and secrete a variety of hydrolytic enzymes (Odds, F. C., J. Am. Acad. Dermatol. 31:S2-5 (1994)). Species within the Candida genus include C. glabrata, C. albicans, C. tropicalis, C. parapsilosis, C. kefir, C. krusei and C. viswanathii.

C. albicans is an important opportunistic pathogen of immunocompromised patients, causing superficial mucocutaneous infections as well as serious systemic infections, which cause almost 30% mortality in these patients (Gale et al, Science 279:135-1358 (1998)). The frequency of systemic fungal infections has increased in the last several decades, largely due to an increase in the number of immunosuppressed patients and patients treated with long-term antibiotic therapy, chemotherapy, and invasive procedures or devices (Georgopapadakou, Curr. Opin. Microbiol. 1:547-557 (1998)). The major responsible pathogen is C. albicans, normally a commensal organism, but the relative fraction of infections from non-albicans Candida species is increasing (Fidel et al, Clin. Microbiol. Rev. 12:80-96 (1999)).

Treatment of fungal infections, such as Candida infections, has lagged behind treatment of bacterial infections. There are numerous commentators who have speculated on this apparent neglect. See, for example, Georgopapadakou et al., Science 264:371 (1994). First, like mammalian cells, fungi are eukaryotes and thus agents that inhibit fungal protein, RNA, or DNA biosynthesis may do the same in the patient's own cells, producing toxic side effects. Second, until recently, life-threatening fungal infections were thought to be too infrequent to warrant aggressive research by the pharmaceutical industry. Other factors have included:

Lack of drugs. The drug, Amphotericin B, has become the mainstay of therapy for fungal infection despite side effects so severe that the drug is known as “amphoterrible” by patients. Only a few second-generation drugs exist.

Increasing resistance. Long-term treatment of oral candidiasis in AIDS and other immune compromised patients has begun to breed species resistant to older anti-fungal drugs. Several other species of fungi have also begun to exhibit resistance.

Lagging research. Because pathogenic fungi are difficult to culture, and because many of them do not reproduce sexually, microbiological and genetic research into disease-causing organisms has lagged far behind research into other organisms.

It is thus apparent that there is a need in the art to improve the panel of anti-fungal agents available to the practitioner to treat fungal infection, such as Candida infection. One way to obtain this goal in Candida is to study how Candida grows and infects its host. Unfortunately, many of the Candida species, such as C. albicans, are diploid, and therefore study of its genome (to determine which genes are essential for growth) is difficult. However, C. glabrata is a haploid organism, and thus study of its genome would be more productive.

An effective way of studying the genome of a Candida species to determine which genes are essential for certain functions is a deletion/complementation test. In a deletion/complementation test, a gene is deleted from the genome of the organism and the organism is studied to determine the role and necessity of the gene in Candida growth and proliferation. To assess complementation, a vector which expresses the deleted gene is introduced into the organism to determine if the gene product returns the organism to its normal phenotype.

Unfortunately, there is a lack of effective vectors which can be used to express genes in Candida. Thus, there is a need in the art for a vector which can effectively express Candida genes in Candida.

### SUMMARY OF THE INVENTION

The present invention provides vectors for expressing a gene of interest in a Candida species. The vectors of the present invention comprise a multiple cloning site; promoter and terminator sequences from a regulatable native Candida gene flanking said multiple cloning site, wherein said gene is a regulated gene; an autonomously replicating sequence; and a centromere sequence. In a preferred embodiment, the vector has both an S. cerivisiae ARS/CEN sequence and a C. glabrata ARS/CEN sequence. In a another preferred embodiment of the present invention, the vector also comprises a gene of interest cloned into the multiple cloning site of the vector. In yet a further preferred embodiment of the present invention, the gene of interest is from a Candida species, preferably the Candida species in which you would like to express the gene of interest. In yet another preferred embodiment of the present invention, the promoter and terminator sequences are from a regulatable native Candida glabrata gene. In another preferred embodiment of the present invention, the regulatable promoter and terminator sequences is from a Candida species, preferably the Candida species in which you would like to express the gene of interest. A regulatable gene is one for which the expression level of the gene can be altered by changes in the growth environment of the cell (e.g., a chemical added to the medium, temperature, etc.). An experimenter can manipulate the expression level by changing growth conditions.

The present invention further provides for a method for complementing deleted genes in Candida species. This method comprises deleting a gene of interest from a Candida species and transforming this Candida species with a vector of the present invention comprising the gene of interest. In a preferred embodiment of the present invention, the gene of interest is deleted from the Candida species by constructing a deletion fragment which replaces the gene to be deleted with a selectable marker and transforming the Candida species with the deletion fragment.

Additionally, the present invention provides for a method for identifying genes essential for the growth of a Candida species. This method comprises deleting a gene from the genome of the Candida species. Once the gene is deleted, the growth of the Candida species which has had a gene deleted is studied and the growth inhibition is assayed. In addition, the deleted gene is complemented using the vector of the present invention to express the deleted gene. Finally, it is determined if expression of the deleted gene restores the Candida species growth.

The present invention also provides for a method for determining a function of a gene from a Candida species. This method comprises deleting a gene from the genome of a Candida species (preferably using the deletion fragments described above). Functional assays are performed determine the effect the deletion has on the growth of the organism. The deleted gene is complemented using the vector of the present invention to express the deleted gene, and then it is determined if expression of the deleted gene restores the function which was lost when the gene was deleted.

The present invention further provides a method for screening active compounds and agents to determine if they have anti-fungal activity on C. glabrata, or any related Candida species. In this aspect of the present invention, essential genes are determined and an overexpression rescue assay is performed. Specifically, in a Candida cell the essential gene is over expressed using the vector of the present invention. To this cell, and to a control cell which expresses just one copy of the essential gene, an active compound is added and the effect the active compound has on the cells is determined by any appropriate method of screening known to one of skill in the art. If the active compound targets the essential gene, or a biochemical pathway requiring the expression product of the essential gene, expressed by the vector of the present invention, then the cell in which the gene is overexpressed will be more resistant to the active compound than the control cell. By these means, novel antifungal compounds can be identified. Furthermore, previously known antifungal compounds can be tested to determine if they are effective in Candida species.

Furthermore, the present invention provides a method of treating a Candida infection in a mammal comprising administering to a mammal a therapeutically effective amount of an anti-fungal compound. The present invention further provides a method of inhibiting the growth of Candida in vivo or in vitro, wherein said method comprises contacting the Candida species with an effective amount of an anti-fungal compound determined to be effective by the methods of the present invention.

Finally, the present invention provides a method for making an antifungal compound, comprising screening for anti-fungal compounds using the method described above and then synthesizing a compound determined to be effective against Candida in an amount sufficient to provide the compound in a therapeutically effective amount to a patient.

Definitions:

The term “active agent,” “antifungal agent” or “antifungal compound” refers to both naturally occurring agents and agents synthesized or modified in the laboratory which may or may not be known to have fungicidal or fungistatic activity. In general, if an antifungal agent is fungistatic, it means that the agent essentially stops fungal cell growth (but does not kill the fungus); if the agent is fungicidal, it means that the agent kills the fungal cells (and may stop growth before killing the fungus). A compound, agent, or composition which is indicated as having antifungal activity exerts an effect on a particular fungal target or targets which is deleterious to the in vitro and/or in vivo growth of a fungal strain containing that target or targets. In particular, an active compound exerts an action which effects the expression product of a target gene. This does not necessarily mean that the compound acts directly on the expression product of the gene, but instead indicates that the compound affects the expression product in a deleterious manner. Thus, the direct target of the compound may be, for example, an upstream component which reduces transcription from the target gene, resulting in a lower level of expression. Likewise, the compound may affect the level of translation of a polypeptide expression product, or may act on a downstream component of a biochemical pathway in which the expression product of the target gene has a major biological role. Consequently, such a compound can be said to be active against the fungal gene, against the fungal gene product, or against the related component either upstream or downstream of that gene or expression product. While the term “active against” encompasses a broad range of potential activities, it also implies some degree of specificity of target. Therefore, for example, a general protease is not “active against” a particular fungal gene which produces a polypeptide product. In contrast, a compound which inhibits a particular enzyme is active against that enzyme and against the fungal gene which codes for that enzyme.

A “coding sequence” or “coding region” refers to an open reading frame (ORF) which has a base sequence which is normally transcribed in a cell (e.g., a fungal cell) to form RNA, which in most cases is translated to form a polypeptide. For the genes for which the product is normally a polypeptide, the coding region is that portion which encodes the polypeptide, excluding the portions which encode control and regulatory sequences, such as stop codons and promoter sequences.

In the context of the coding sequences and genes of this invention, “homologous” refers to genes whose expression results in expression products which have a combination of amino acid sequence similarity or identity (or base sequence similarity for transcript products) and functional equivalence, and are therefore homologous genes. In general such genes also have a high level of DNA sequence similarity (i.e., greater than 80% when such sequences are identified among members of the same genus, but lower when these similarities are noted across fungal genera), but are not identical. Preferred genetic homologs include those genes which are about at least 85%, 90% or 95% similar at the nucleic acid or the amino acid level. The combination of functional equivalence and sequence similarity means that if one gene is useful, e.g., as a target for an antifungal agent, or for screening for such agents, then the homologous gene is likewise useful. In addition, identification of one such gene serves to identify a homologous gene through the same relationships as indicated above. Typically, such homologous genes are found in other fungal species, especially, but not restricted to, closely related species.

Due to the DNA sequence similarity, homologous genes are often identified by hybridizing with probes from the initially identified gene under hybridizing conditions which allow stable binding under appropriately stringent conditions (e.g., conditions which allow stable binding with at least approximately 85% or more sequence identity). Hybridization methods are known in the art and include, but are not limited to: (a) washing with 0.1× SSPE (0.62 M NaCl, 0.06 M NaH₂PO₄.H₂O, 0.075 M EDTA, pH 7.4) and 0.1% sodium dodecyl sulfate (SDS) at 50° C.; (b) washing with 50% formamide, 5× SSC (0.75 M NaCl, 0.075 M sodium citrate), 50 mM sodium phosphate (pH 6-8), 0.1% sodium pyrophosphate, 5× Denhardt's solution, sonicated salmon sperm DNA (50 μg/ml), 0.1% SDS and 10% dextran sulfate at 42° C., followed by washing at 42° C. in 0.2× SSC and 0.1% SDS; and (c) washing with of 0.5 M NaPO₄, 7% SDS at 65° C. followed by washing at 60° C. in 0.5× SSC and 0.1% SDS. High stringency hybridization conditions are those performed at about 20° C. below the melting temperature (Tₘ) of the probe. Preferred stringency is performed at about 5-10° C. below the melting temperature (Tₘ) of the probe. Additional hybridization conditions can be prepared as described in Chapter 11 of Molecular Cloning: A Laboratory Manual, 2nd Ed., ed. By Sambrook, Fritsch and Maniatis (Cold Spring Harbor Laboratory Press: 1989), or as would be known to the artisan of ordinary skill. The equivalent function of the product is then verified using appropriate biological and/or biochemical assays.

Alternatively, Candida libraries can be probed using degenerate primers and polymerase chain reaction (PCR) techniques to identify variants of the gene identified originally. Preferably, primers are utilized which hybridize under stringent conditions to the open reading frame of the gene identified originally. Preferred primers are typically 15 nucleotides in length, but can vary to be at least, about 16, 17, 18, 19, 20, 21, 22, 23, 24, 25, 30, 35 or 40 nucleotides in length.

By a polypeptide having an amino acid sequence of at least, for example, 95% “identity” to a reference amino acid sequence or fragment thereof is intended that the amino acid sequence of the polypeptide is identical to the reference sequence except that the polypeptide sequence may include up to five amino acid alterations per each 100 amino acids of the reference amino acid sequence. In other words, to obtain a polypeptide having an amino acid sequence 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Alignment programs can be used to identify conserved sequences or potential motifs across different animal species. Alignment programs can also be used to align the nucleic acid and/or protein sequences of related genes and the proteins that they encode. Preferred alignment programs include CLUSTALW, PILEUP and GAP, and would preferably be used with default parameters.

By a polynucleotide having a nucleotide sequence at least, for example, 90% “similar” to a reference nucleotide sequence encoding a polypeptide, is intended that the nucleotide sequence of the polynucleotide is identical to the reference sequence except that the polynucleotide sequence may include up to ten point mutations per each 100 nucleotides of the reference nucleotide sequence The term “fungal gene product” or “expression product” is used to refer to a polypeptide or RNA molecule which is encoded in a DNA sequence according to the usual transcription and translation rules, which is normally expressed by a fungus. Thus, the term does not refer to the translation of a DNA sequence which is not normally translated in a fungal cell. However, it should be understood that the term does include the translation product of a portion of a complete coding sequence and the translation product of a sequence which combines a sequence which is normally translated in fungal cells translationally linked with another DNA sequence. The gene product can be derived from chromosomal or extrachromosomal DNA, or even produced in an in vitro reaction. Thus, as used herein, an “expression product” is a product with a relevant biological activity resulting from the transcription, and usually also translation, of a fungal gene.

The term “inhibiting the growth” indicates that the rate of increase in the numbers of a population of a particular fungus is reduced. Thus, the term includes situations in which the fungal population increases but at a reduced rate, as well as situations where the growth of the population is stopped, as well as situations where the numbers of the fungus in the population are reduced or the population even eliminated. The term also includes situations in which the fungus is killed.

The term “method of screening” means that the method is suitable, and is typically used, for testing for a particular property or effect in a large number of compounds.

The term “therapeutically effective amount” means a concentration of active agent at the site of infection or in the bloodstream which is effective to inhibit fungal growth. This concentration can be determined from MIC values obtained from in vitro growth inhibition studies, using known human pathogenic fungi, and is related to the fungus type. Thus, for many applications, an effective dose is preferably one which results in a concentration of active antifungal compound in this range at the site of fungal infection.

The term “derived from” a known gene or protein means that the gene or protein is the native known gene or protein, or is a gene or protein which is derived therefrom and has a significant amount of homology with said known gene or protein so that it has the same function as said known gene or protein. Preferably, a gene or protein derived from a known gene or protein should share at least about 80% similarity with said known gene or protein, preferably at least 85%, and more preferably at least 90% or 95% similarity.

The term “mammal” refers to any organism of the Class Mammalia of higher vertebrates that nourish their young with milk secreted by mammary glands, e.g., mouse, rat, and, in particular, human, dog, and cat.

The term “resistance,” as it refers to the effect of an anti-fungal compound on a fungus, refers to how much the anti-fungal compound effects fungal growth and cellular proliferation. For example, when a fungus has high resistance to an anti-fungal compound, the anti-fungal compound has little or no effect on fungal growth and cellular proliferation. In contrast, when a fungus has low resistance to an anti-fungal compound, the anti-fungal compound has a deleterious effect on fungal growth (by slowing growth) and cellular proliferation (by slowing cellular proliferation).

The practice of the present invention will employ, unless otherwise indicated, conventional techniques of cell biology, cell culture, molecular biology, transgenic biology, microbiology, and recombinant DNA, which are within the skill of the art. Such techniques are explained fully in the literature. See, for example, Molecular Cloning: A Laboratory Manual, 2nd Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; B. Perbal, A Practical Guide to Molecular Cloning (1984).

### BRIEF DESCRIPTION OF THE DRAWINGS

FIG. 1A. Construction of the deletion fragments—Position of primers used to construct the deletion fragment. The black box depicts the C. glabrata HIS3 gene including its upstream and downstream flanking sequences. The unshaded and gray boxes represent sequences flanking and within the coding sequence of YFG (“Your Favorite Gene”), the gene to be deleted. The inner-left and inner-right primers are hybrid primers with some sequence derived from HIS3 and some from YFG, as depicted by the shading.

FIG. 1B. Construction of the deletion fragments—The three individual PCR fragments generated in the initial PCR reaction.

FIG. 1C. Construction of the deletion fragments—The final PCR product, which results from a PCR reaction containing the left and right primers with the left flank, HIS3, and right flank PCR products serving as template after denaturation during the PCR reaction. This final PCR product was used to transform C. glabrata to generate the deletion strains.

FIG. 1D. Construction of the deletion fragments—A comparison of the YFG sequences remaining in different stages of the experiments described herein. The top line represents the wild-type YFG locus in the chromosome with the coding sequence in gray. The middle line represents the YFG locus in the deletion strain (some wild-type sequences remain). The bottom line shows the extent of the wild-type gene carried on the expression plasmid (the entire coding sequence).

FIG. 2. Map of the pFPG1 (Fungal Plasmid GTC No. 1) expression plasmid. “P CgMT-1” and “T CgMT-1” represent the promoter and terminator, respectively, from the C. glabrata metallothionein I gene. Restriction sites marked are unique to the plasmid. Sc represents S. cerevisiae-derived sequences.

FIG. 3. Map of the pFPG2 (Fungal Plasmid GTC No. 2) expression plasmis. “P CgMT-1” and “T CgMT-1” represent the promoter and terminator, respectively, from the C. glabrata metallothionein I gene. Restriction sites marked are unique to the plasmid. Sc represents S. cerevisiae-derived sequences. Cg represents C. glabrata derived sequences.

FIG. 4. Growth phenotypes of C. glabrata strains carrying the expression plasmid pFPG1. To evaluate the expression vector, growth of strains carrying the expression plasmid pFPG1 with or without a wildtype test gene were compared on selective media lacking or containing copper sulfate to induce the MT-I promoter. All strains were growth for three days.

A. C. glabrata Δade2 strain with expression plasmid pFPG1 lacking or carrying the wildtype ADE2 gene, on -His -Ura -Ade media.

B. C. glabrata Δleu2 strain with expression plasmid pFPG1 lacking or carrying the wildtype LEU2 gene, on -His -Ura -Leu media.

C. C. glabrata strain with expression plasmid pFPG1 lacking or carrying the wildtype neomycin/kanamycin resistance gene (which confers resistance to the antibiotic geneticin), on -Ura media supplemented with 1 mg/ml geneticin.

FIG. 5. Growth phenotypes of C. glabrata strains carrying the C. glabrata CEN-ARE expression plasmid pFPG2. To evaluate the expression vector, growth of strains carrying the expression plasmid pFPG2 with or without a wildtype test gene were compared on selective media lacking or containing copper sulfate to induce the MT-1 promoter. All strains were grown for three days.

A. C. glabrata Δade2 strain with expression plasmid pFPG2 lacking or carrying the wildtype ADE2 gene, on -His -Ura -Ade media.

B. C. glabrata Δleu2 strain with expression plasmid pFPG2 lacking or carrying the wildtype LEU2 gene, on -His -Ura -Leu media.

C. C. glabrata strain with expression plasmid pFPG2 lacking or carrying the wildtype neomycin/kanamycin resistance gene (which confers resistance to the antibiotic geneticin), on -Ura media supplemented with 1 mg/ml geneticin.

### DETAILED DESCRIPTION OF THE INVENTION

A major object of the present invention is to provide a system for complementing a deleted gene in C. glabrata and related Candida species. As mentioned previously, there is a lack of expression vectors which are useful for expressing genes in C. glabrata. In fact, prior to the present invention no expression vector is known to the inventors which uses a regulatable promoter and terminator from a C. glabrata gene.

An exemplary vector of the present invention was constructed by cloning the promoter and terminator sequences from the C. glabrata MT-1 metallothionein gene into restriction enzyme sites flanking the multiple cloning site of the plasmid, pRS416. The vector may also contain the C. glabrata ARS10 and CEN8 regions, which allow for stable maintenance of the vector in low copy number. Although the MT-1 promoter and terminator sequences were used in the vector exemplified herein, this is just exemplary; one of skill in the art would know of other promoter and terminator sequences from other Candida genes which have regulated expression, which could be used in the vector of the present invention. For example, the promoter and terminator sequences from any of the following C. glabrata genes could be used in the vector of the present invention: TRPI, HIS3, ADE2, LEU2, and URA3. Promoters and terminators which are homologous to known Candida promoters and terminators can also be used.

Furthermore, the use of the plasmid, pRS416, is also just exemplary. Other plasmids known to one of skill in the art can also be used in the present invention. The plasmids of the present invention should comprise a replication origin, preferably ARS-CEN, which is comprised of an S. cerevisiae autonomously replicating sequence (for example, S. cerevisiae ARSH4 or C. glabrata ARS10, but one of skill in the art would know of other autonomously replicating sequences) and a centromere sequence (for example, S. cerevisiae CEN6 or the CEN8 region from C. glabrata (Kitada et al., Gene 165:203-206 (1995)); and a multi-cloning site. In a preferred embodiment of the present invention, the ARS-CEN region is from a Candida species. For C. glabrata, a preferred ARS sequence is ARS10 and a preferred Cen sequence is CEN8. Preferably, the plasmids of the present invention should also comprise a selectable marker (such as URA3, HIS3, LEU2 and TRP1, or other known selectable markers) and an E. coli antibiotic resistance gene (such as amp, tetracycline, chloramphenicol, and kanamycin, or other known E. coli antibiotic resistance genes).

The vector of the present invention can be used to express any gene, either foreign or native to C. glabrata. Furthermore, the vector of the present invention is not limited to use in just C. glabrata. For example, the vector of the present invention can potentially be used to express genes, either foreign or native, in other related Candida species, such as C. albicans, C. tropicalis, C. parapsilosis, C. kefir, C. krusei and C. viswanathii.

Additionally, more than one gene can be expressed simultaneously using the vector of the present invention. The advantage of expressing more than one gene simultaneously is that you can complement two or more gene deletions at the same time.

The preferred vectors allow regulatable expression of a gene cloned into them (gene expression requires induction by growth in the presence of copper). In a strain deleted for a gene of interest, this would be useful for easy comparison of growth, transcription, or other properties in the presence or absence of the protein of interest. The vector would also be useful in experiments that require transient expression of a protein of interest (e.g. for expression of a deleterious protein only when needed, or for simultaneous production of a “burst” of protein). Finally, down-regulation of expression in a strain deleted for a gene of interest can be used to test for essentiality of the gene encoding the protein. Furthermore, down-regulation of an essential gene can be used to test whether lack of a protein causes a cidal phenotype (cell death) or a static phenotype (inhibition of cell growth).

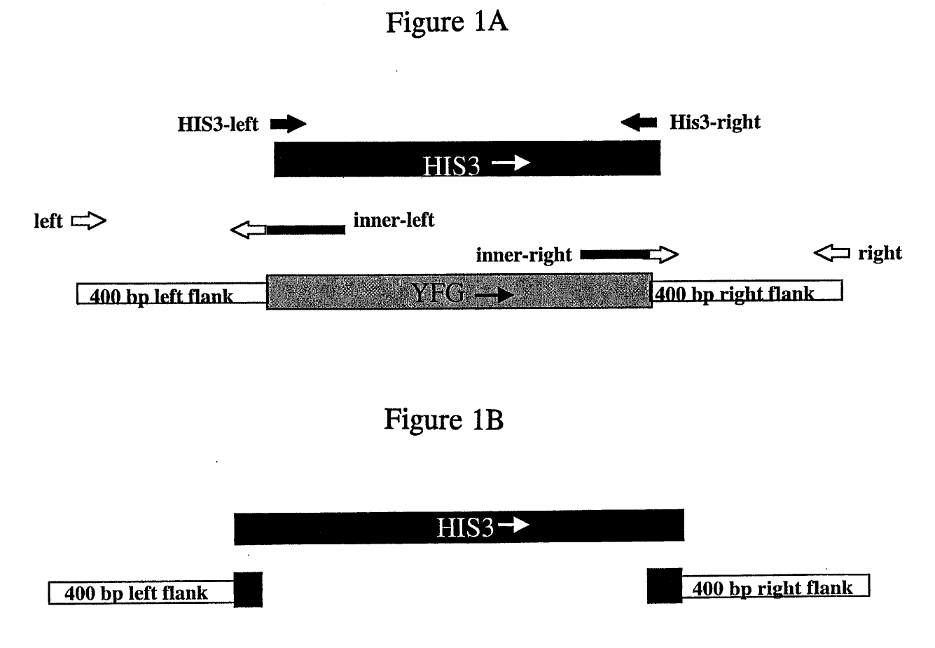

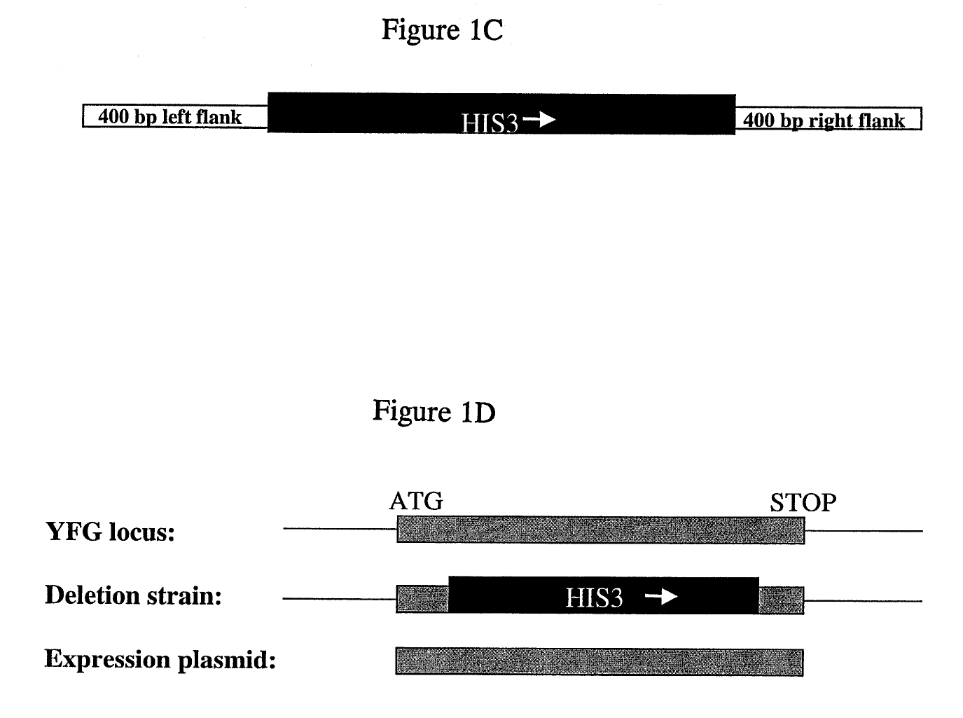

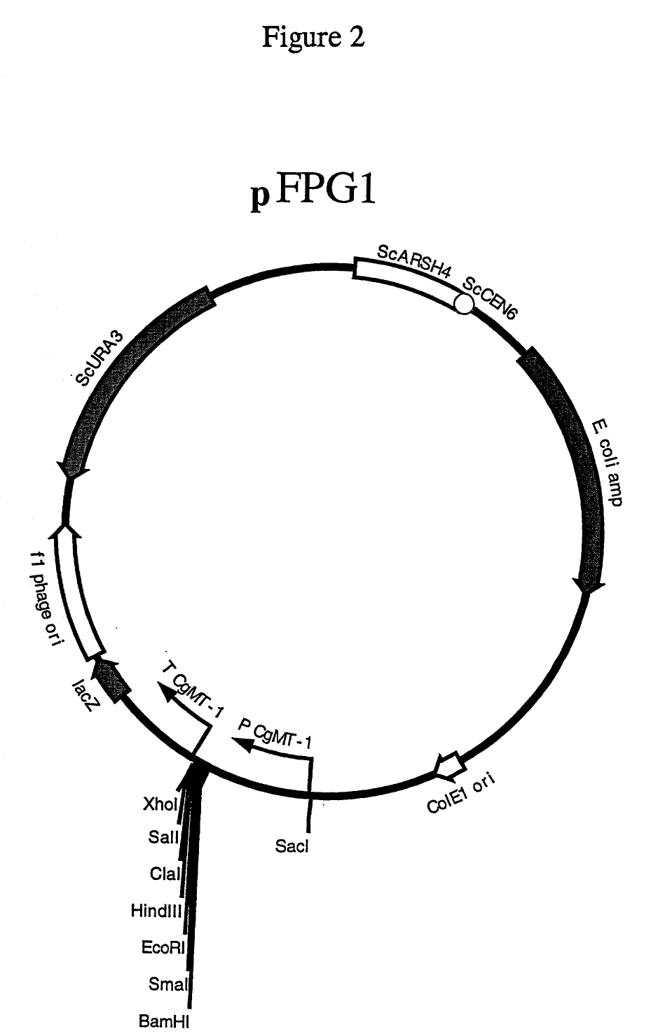

Another object of the present invention is to provide a method for deleting a fungal gene of interest, whether it be non-essential or essential, from C. glabrata or a related Candida species and subsequently expressing the gene in the organism using the vector of the present invention. This method involves replacing a gene of interest with another gene, for example C. glabrata HIS3 gene, by crossover PCR or SOEing (splicing by overlap extension). This process is described in FIG. 1 and in Example 1. Also, the following references describe this or related techniques. G. Pont-Kingdon, BioTech. 16:1010-1011 (1994); R. M. Horton, Methods in Molecular Biology, Vol. 67: PCR Cloning Protocols: From Molecular Cloning to Genetic Engineering, Ed. B. A. White, Humana Press, Inc., Totowa, N.J. (1991), pp. 141-149; and B. Lefebvre et al, BioTech. 19:186-187 (1995). First, hybrid primers are developed which contain some sequence derived from the coding region of the replacement gene and some sequence from the gene of interest (either coding or non-coding). These primers are used to amplify 400 bp upstream and downstream of the gene of interest, and the end products are: 1) a fragment which contains 400 bp upstream of the gene of interest and sequence from the 5′ region of the replacement gene (the left flank); and 2) a fragment which contains 400 bp downstream of the gene of interest and sequence from the 3′ region of the replacement gene (the right flank). See, for example, FIG. 1. These two fragments, together with a fragment which contains the replacement gene, are subjected to crossover PCR or SOEing using left and right primers which bind to the ends of the left and right flanks, respectively. The final product fragment contains the 400 bp left flank, the replacement gene, and the 400 bp right flank. This final product (the “deletion fragment”) is transformed into the organism and, by a crossover recombination reaction, will delete the gene of interest from the organism's chromosome. For the deletion of multiple genes, multiple deletion fragments are produced, each specifically made for the deletion of a different gene of interest.

Using this method, any non-essential gene can be deleted. However, in order to delete essential genes, it is necessary to express the wild-type gene in the cells undergoing the deletion process (since the essential gene is needed for the growth and survival of the organism). Thus, the present invention also provides a method of complementing a gene deleted from a Candida species by controllably expressing the wild-type gene in the species using the vector of the present invention.

Another object of the present invention is to provide a method for determining which genes in C. glabrata, or any related Candida species, are essential for organism growth or cell proliferation and what the function of the expression products of these genes are. This method of the present invention involves deleting a gene of interest (as discussed in more detail above) and determining if the deletion inhibits the growth of the fungus. If growth is inhibited, then one would re-introduce the wild-type gene using the vector of the present invention to express it. If growth is restored, then the gene deleted is essential for growth of the Candida species tested.

To determine the function of a gene, the gene is deleted from the genome of a Candida species (preferably using the deletion fragments described above). Subsequently, functional assays are performed to determine the effect the deletion has on the growth of the organism. The deleted gene is complemented using the vector of the present invention to express the deleted gene, and then it is determined if expression of the deleted gene restores the function which was lost when the gene was deleted.

Once an essential gene is identified in one Candida species, this gene can be used to identify homologous essential genes in other Candida species. Once a homologous gene is identified, it can be determined, using the method described above, whether the gene is an essential gene in the Candida species being tested.

A further object of the present invention is to provide a method for screening active compounds and agents to determine if they have anti-fungal activity on C. glabrata, or any related Candida species. In this aspect of the present invention, essential genes are determined as discussed above and an overexpression rescue assay is performed. Specifically, in a Candida cell the essential gene is overexpressed using the vector of the present invention. To this cell, and to a control cell which expresses just one copy of the essential gene, an active compound is added and the effect the active compound has on the cells is determined by any appropriate method of screening known to one of skill in the art. If the active compound targets the essential gene, or a biochemical pathway requiring the expression product of the essential gene, expressed by the vector of the present invention, then the cell in which the gene is overexpressed will be more resistant to the active compound than the control cell. By these means, novel antifungal compounds can be identified. Furthermore, previously known antifungal compounds can be tested to determine if they are effective in Candida species.

Yet another object of the present invention is to provide a method for treating a fungal infection in a mammal by administering to a mammal or prophylactically treating a mammal with an amount of an anti-fungal agent effective to reduce the infection. The antifungal agent specifically inhibits a biochemical pathway requiring the expression product of a gene corresponding to one of the genes identified by the methods of the present invention, or inhibits the expression product of the gene itself, and thereby inhibits the growth of the fungus in vivo. In particular embodiments, the antifungal agent inhibits the expression product of one of the identified genes.

Another aspect of the present invention provides a method of inhibiting the growth of a pathogenic fungus of the Candida species in vivo or in vitro by contacting the fungus with an antifungal agent which specifically inhibits a biochemical pathway requiring the expression product of a gene identified by the method of the present invention as being an essential gene for growth. Inhibition of that pathway inhibits growth of the fungus. In particular embodiments, the antifungal agent inhibits the expression product of one of the identified genes.

The present invention further provides for a method for making an antifungal compound. Once an compound is identified as being an antifungal agent, the agent is synthesized in an amount sufficient to provide said agent in a therapeutically effective amount to a patient.

All of the references and patents cited herein are incorporated by reference.

The following examples are presented in order to more fully illustrate the preferred embodiments of the invention. They should in no way be construed, however, as limiting the broad scope of the invention.

### EXAMPLE 1

High-Throughput Method for Disruption of Candida glabrata Genes

The chromosomal copy of the C. glabrata ADE2 and C. glabrata LEU2 genes were deleted using a PCR-based technique to generate a linear deletion fragment in which the gene of interest was replaced by the C. glabrata HIS3 gene (see FIG. 1). The deletion fragment was generated by crossover PCR or SOEing (splicing by overlap extension) from three pieces: 1) a fragment containing 400 bp of sequence derived from the region upstream of the gene of interest, with a short 40 base segment of homology to the 5′ end of the HISS marker; 2) the HIS3 marker (sequences from ATG −223 bp to STOP +171 bp); and 3) a fragment containing 400 bp of downstream sequence from the gene of interest, with a short 40 base segment of homology to the 3′ end of the HISS marker. Each of these three pieces was generated by a PCR reaction using C. glabrata genomic DNA from strain ATCC No. 200989 (ADE2 and LEU2 fragments) or ATCC No. 38326 (HISS fragment) (Table 1) as the template and with the primers shown in FIG. 1 and Table 3. The three fragments were purified on a Qia-quick™ column (Qiagen) and used as a template in a final PCR reaction with only the two outermost primers (left and right) to generate a fragment containing the HISS marker gene flanked by 400 bp segments upstream and downstream of the gene of interest. As numbered relative to the ATG translation start site, the ADE2 deletion fragment would remove sequence from +240 bp to +1497 bp (215 bp before the STOP codon) or about 74% of the ORF and the LEU2 deletion fragment would remove sequence from −20 bp to +852 bp (245 bp before the STOP codon) or about 78% of the ORF. The DNA was concentrated by ethanol precipitation and approximately 30 ug was used to transform C. glabrata strain ATCC No. 200989 (Δhis3, Δtrp1, Δura3). HisA⁺ transformants were selected, patched onto -His media, and tested for deletion of the ADE2 or LEU2 genes by their growth phenotype after replica-plating onto selective media (-His -Ade or -His -Leu media). It was discovered that about 20% of the transformants had the growth phenotype expected for a deletion of the chromosomal locus (Ade⁻ or Leu⁻, respectively) (Table 2). Genomic DNA was prepared from five transformants of each type (Ade⁻, Ade⁺, Leu⁻, and Leu⁺) and used in a PCR test to assess whether the deletion fragments had integrated in the chromosome and replaced the wild-type locus as expected. In each case, all transformants expected to have the deletion by their growth phenotype also showed correct integration by the PCR test and none of the transformants with a wild-type phenotype did. This indicated that the deletion fragment recombined in a double crossover event with the homologous locus about 20% of the time. One Δade2 strain (FSGO15) and one Δleu2 strain (FSG020) were chosen for further study.

This method can easily be adapted for high-throughput deletion of multiple C. glabrata genes. Any non-essential gene can be deleted directly. However, to delete essential genes, it is necessary to express the wild-type gene in the cells undergoing the deletion process. Toward that end, a plasmid was constructed that would allow expression of a wild-type copy of C. glabrata genes.

TABLE 1
C. glabrata strains used

| Name | Source | Genotype |
|---|---|---|
| ATCC 200989 | ATCC, Kitada et al. | Δhis3, Δtrp1, Δura3 |
| | (1995) | |
| ATCC 38326 | ATCC | |
| FSG015 | This work | ATCC 200989, Δade2::HIS3 |
| FSG020 | This work | ATCC 200989, Δleu2::HIS3 |

TABLE 2
Results of deletion experiment. Individual transformants (trf's) were
replica-plated onto selective media and nonselective media (e.g., -Ade
-His and -His media for the ADE2 deletion).

| | No. (%) of trf's that fail to grow | |
|---|---|---|
| | on selective media (out of #100 | |
| Gene deleted | transformants tested) | PCR test results |
| ADE2 | 30% (first experiment) | 5/5 Ade have deletion |
| | 11% (second experiment) | |
| LEU2 | 28% (first experiment) | 5/5 Leu have deletion |
| | 15% (second experiment) | |

TABLE 3
Primers used to generate the three fragments of DNA used to generate
the deletion fragment.

| Primer | Sequence |
|---|---|
| CgADE2-left | ACT GTG TGT CAA GTT CCA ATT GAG |
| CgADE2-CgHIS inner left | GTC TTT TCT GAG GAA TTT CAA GTT |
| | TCT GAG TCA TTT TCC TCG CCA ATG |
| | CAT TAA CAT CCA CAT GCT CGA |
| CgADE2-CgHIS inner right | TGT GCC ATT CAT AAA CGT GAT CAC |
| | TTT ACG TAG CAG GCA ACC CCA CTA |
| | TTG TTC AAA TGC CAA GAG GTG |
| CgADE2-right | CTA CGT GTT ACA CTG GAA TGA AGG |
| CgLEU2-left | CCC TAT CTT GAA ACT GGT TAT GGT |
| CgLEU2-Cg HIS inner left | GTC TTT TCT GAG GAA TTT CAA GTT |
| | TCT GAG TCA TTT TCC TCG CCA GTG |
| | TGT GTA TAG TGT ATC CTC TTC |
| CgLEU2-CgHIS inner right | TGT GCC ATT CAT AAA CGT GAT CAC |
| | TTT ACG TAG CAG GCA ACC CCA TAT |
| | GAG CCA TGT CAT GGC TCT GCT |
| CgLEU2-right | CAG ATG ATT CAC CGG TTT GAT AGT |
| CgHIS3-left | TGG CGA GGA AAA TGA CTC AGA |
| | AAC TTG |
| CgHIS3-right | TGG GGT TGC CTG CTA CGT AAA GTG |
| CgHIS3-B | TGC TCT CCC GTA CAG AAA CAG ACC |
| CgHIS3-C | CCA TCG CCA TCA GAG AGG CAA |
| | GAA |
| CgADE2-A | TGA AAG ATA CTT TTC TGC CAC TCC |
| CgADE2-D | GAT GAC TAT CAG AAA TTT GGC GTT |
| | GAT |
| CgADE2-E | GTA GGG TAG ATT TCC AAT TTG GGG |
| CgADE2-F | GAG TAC CTG TCA AGG GCT CAT TCT |
| CgLEU2-A | CCA ATT CTG TGT TTC CCG GAA ATG |
| CgLEU2-D | GTT CGT TTG CCG ATA CAT GCG AAT |
| CgLEU2-E | TCA CCT GGT GGA ACT ACA ATT GTC |
| CgLEU2-F | TAC TTC CAT CTG CCT CCT TGG CAT |
| P CgMT-1 up | AGG TCC CCG CGG GGA CGC CCT TCA |
| | TAC ACA TCC TAC ACT |
| P CgMT-1 down | AGG TCC CCG CGG GGA TGT GTT TGT |
| | TTT TGT ATG TGT TTG TTG |
| T CgMT-1 up | CAA CGG GGT ACC CCG TTG CAT TAA |
| | CAA CTA AAG CAA ACT ACT |
| T CgMT-1 down | CAA CGG GGT ACC CCG TTT CGT CGT |
| | GGA AGC GTC GAT CGT |
| CgCEN up | AGG TCC GCC GGC GGA TCT AGA |
| | AAA TAC ATA GTG AAT CT |
| CgCEN down | GTC AAA ATG ATC AAG CTT CCG TTG |
| | ACG CGT AAC TAA TGA TGA TGC AAT |
| | TTT TG |
| CgARS up | GCA TCA TTA GTT ACG CGT CAA CGG |
| | AAG CTT GAT CAT TTT GAC CCC ATC |
| CgARS down | AGG TCC GCC GGC GGA CTT ACG CTC |
| | TAT CCG GTA ACG TTA G |
| CgADE2-ATG | GAA CCG CTC GAG CGG ATG GAC |
| | TCT AGA ACT GTC GGT ATT |
| CgADE2-STOP | GAA CCG CTC GAG CGG CTA TTT GGA |
| | CTC TAG GTA CTT TTC |
| CgLEU2-ATG | CAA CGC GGA TCC GCG ATG GCT |
| | GTG ACC AAG ACA ATT GTA |
| CgLEU2-STOP | CAA CGC GGA TCC GCG CTA AGC |
| | TAA TAG TTC CCT GAC AGC |
| neo-ATG | CAA CGC GGA TCC GCG ATG AGC |
| | CAT ATT CAA CGG GAA ACG |
| neo-STOP | CAA CGC GGA TCC GCG TTA GAA |
| | AAA CTC ATC GAG CAT CAA ATG |
| | AAA CTG C |

### EXAMPLE 2

Construction of a Candida glabrata expression plasmids To construct the expression plasmid pFPG2, the promoter and terminator regions of the C. glabrata metallothionein I gene, MT-1, (GenBank accession number J05133) (Mehra et al, J. Biol. Chem. 264:19747-19753 (1989)) and the C. glabrata CEN8 and ARS10 regions (GenBank accession numbers U43926 and U43925) (Kitada et al, Gene 175:105-108 (1996)) were cloned into the S. cerevisiae vector pRS416 (GenBank accession number U03450) (Sikorski and Hieter, Genetics 122:19-27 (1989)), as follows. The promoter region of MT-1 (bases −308 to −1 relative to the ATG translation start) was amplified by PCR with hybrid primers containing the SacII restriction enzyme site (P CgMT-1 up and P CgMT-1 down) using genomic DNA from strain ATCC 38326 and cloned into the SacII site of pRS416. In a similar manner, the terminator region of MT-1 (bases +1 to +253 relative to the STOP codon) was amplified with hybrid primers containing the kPnI restriction site (T CgMT-1 up and T CgMT-1 down) and cloned into the KpnI site of pRS416, at the opposite end of the multiple cloning site. Proper amplification and construction were verified by DNA sequencing across both inserts. This intermediate vector was designated pFPG1.

To generate pFPG2, the C. glabrata CEN-ARS region was cloned into pFPG1. The CEN-ARS region was generated by PCR from two pieces: 1) a fragment containing CgCEN8, with an NgoMIV restriction enzyme site at the 5′ end and a short 18 base segment of homology to CgARS10 at the 3′ end, and 2) a fragment containing CgARS10, with a short 18 base segment of homology to CgCEN8 at the 5′ end and an NgoMIV restriction enzyme site at the 3′ end. These two pieces were generated by PCR amplification with C. glabrata genomic DNA from strain ATCC 38326 using primers CgCEN up and CgCEN down or CgARS up and CgARS down, respectively (Table 1). To generate a fragment containing the entire CEN-ARS region, the two fragments were used as templates in a final PCR reaction with only the two outermost primers (CgCEN up and CgARS down). This assembled CEN-ARS region was cloned into the NgoMIV site of pFPG1 to generate pFPG2. Proper amplification and construction were verified by DNA sequencing across the insert.

### EXAMPLE 3

Expression of Test Genes

Several genes were cloned into pFPG1 to test the function of the plasmid in expression and complementation: the C. glabrata ADE2 and LEU2 genes, and a neomycin/kanamycin resistance gene. It was expected that the ADE2 and LEU2 genes expressed on pFPG1 would complement the adenine- or leucine-dependent growth of the deletion strains constructed previously, ideally in a copper-dependent manner. We amplified the ADE2 gene from its ATG to STOP codons using genomic DNA from ATCC 38326 as the template and using hybrid primers containing the XhoI restriction site and cloned the gene into the XhoI site of pFPG1. The ADE2 pFPG1 plasmid carries a GTG valine codon instead of the expected GTA valine codon at amino acid position 231 (additional changes as compared to the GenBank sequence, accession no. AF030388, are shared between two independent clones and are probably due to strain differences or errors in the GenBank sequence). An ADE2 version of pFPG2 was constructed by cloning the CEN-ARS NgoMIV fragment from pFPG2 into the ADE2 pFPG1 plasmid, selecting a clone with the CEN-ARS fragment in the same orientation as in pFPG2.

Similarly, the LEU2 gene from the ATG to the STOP codons was cloned into the BamHI site of pFPG1 and the LEU pFPG1 plasmid carries no mutations compared to the expected sequence (additional changes as compared to the GenBank sequence, Accession No. U90626, are shared between four independent clones and are probably due to strain differences or errors in the GenBank sequence). A LEU2 version of pFPG2 was constructed by cloning the CEN-ARS NgoMIV fragment from pFPG2 into the LEU2 pFPGl plasmid, selecting a clone with the same CEN-ARS orientation as in pFPG2.

The neomycin/kanamycin resistance gene from Tn903 confers resistance of C. glabrata to the antibiotic G418, geneticin (Cormack and Falkow, Genetics 151:979-987 (1999)). This gene was amplified from the ATG to the STOP codon using plasmid pNK2887 DNA as template with hybrid primers containing the BamHI restriction site and cloned the ORF into the BamHI site of pFPG1. The NEO pFPG1 plasmid carries a leucine codon instead of an isoleucine codon at position 4 compared to the expected sequence reported in GenBank accession no. X06402. A NEO version of pFPG2 was constructed by cloning the CEN-ARS NgoMIV fragment from pFPG2 into the NEO pFPG1 plasmid, selecting a clone with the same CEN-ARS orientation as in pFPG2.

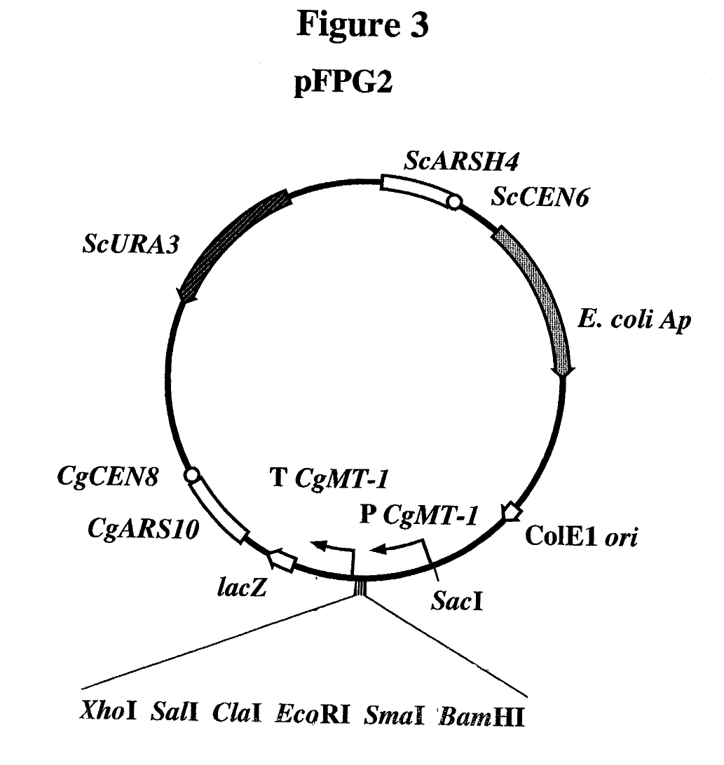

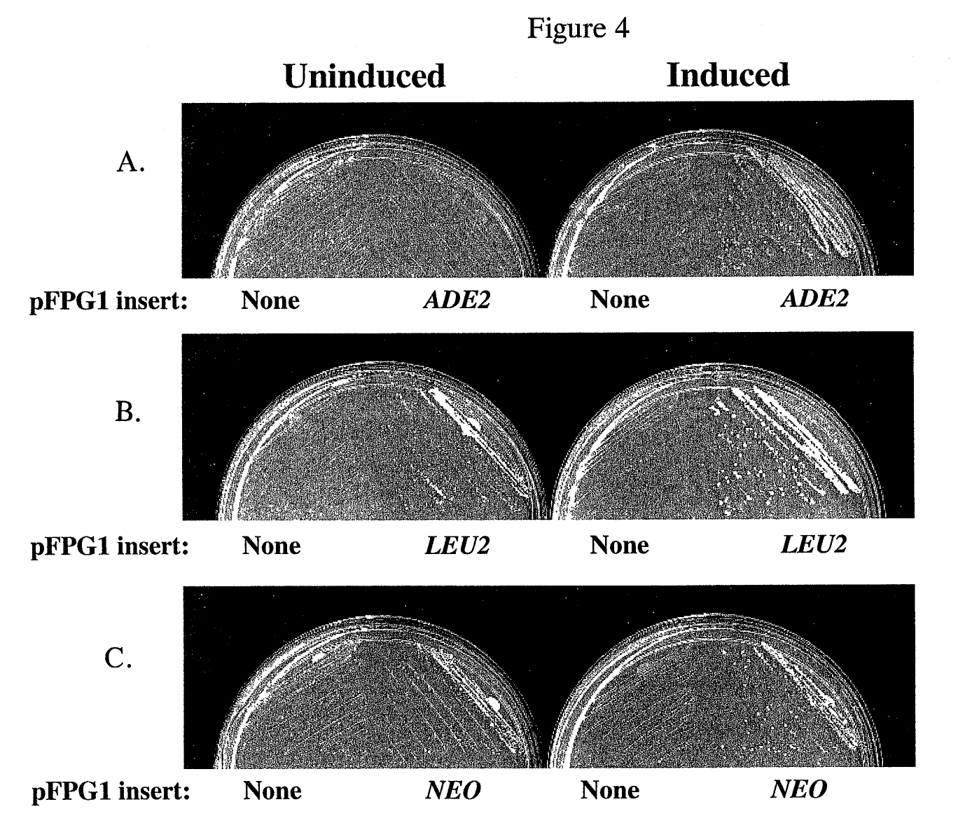

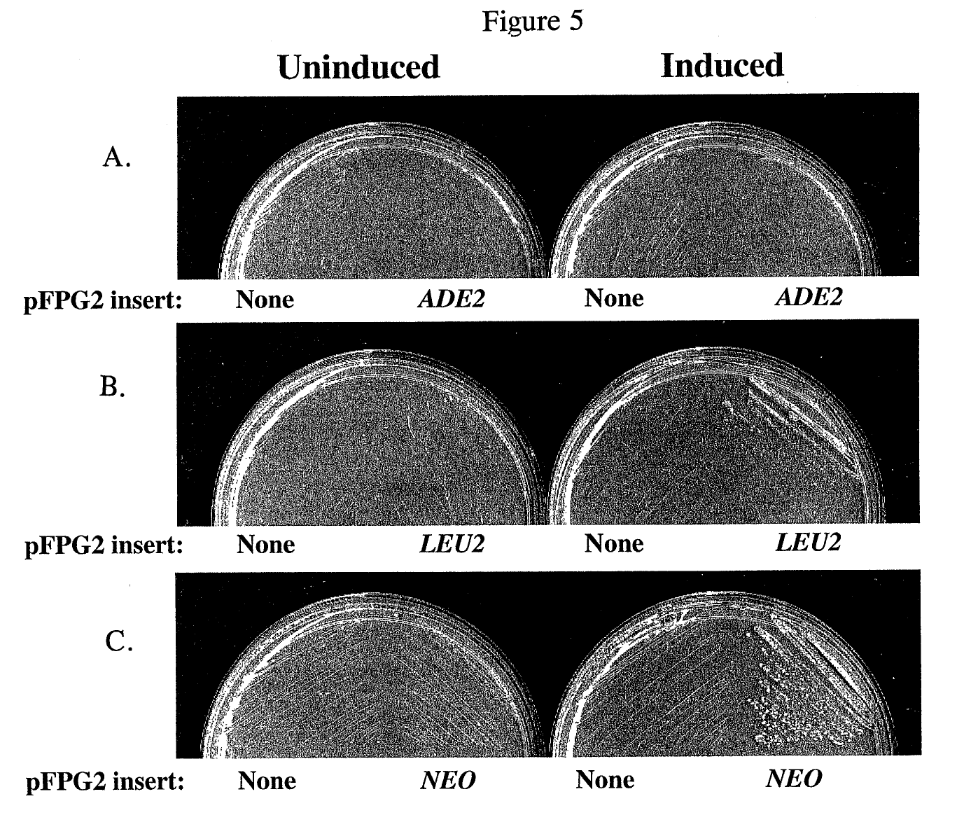

The ADE2 pFPG1 plasmid (or empty pFPG1 as a control) was transformed into strain FSGO15, the C. glabrata Δade2 strain which was constructed, and tested for its ability to complement the adenine-dependent growth of that strain (FIG. 3A). It was found that strain FSG015 carrying the empty plasmid failed to grow either in the presence or absence of copper. Strain FSG015 carrying the ADE2 plasmid failed to grow without copper but grew well in the presence of 0.2 mM copper. This indicated that ADE2 was properly expressed from pFPG1 and that induction of the MT-1 promoter by copper was necessary for its expression.

The LEU pFPG1 plasmid (or empty FPG1 as a control) was transformed into strain FSG020, the C. glabrata Δleu2, strain which was constructed, and tested for its ability to complement the leucine-dependent growth of that strain (FIG. 3B). It was found that strain FSG020 carrying the empty plasmid failed to grow either in the presence or absence of copper. Strain FSG020 carrying the LEU2 plasmid grew moderately well without copper and well in the presence of 0.2 mM copper. This indicated that LEU2 was properly expressed from pFPG1 but that induction of the MT-1 promoter by copper in this case was not absolutely necessary for its expression. Presumably a low level of expression is sufficient to complement the leucine deficiency of strain FSG020.

The neomycin/kanamycin resistance gene from Tn903 confers resistance of C. glabrata to the antibiotic G418, geneticin (Cormack and Falkow, 1999). The pFPG1-NEO plasmid and empty pFPG1 as a control were transformed into C. glabrata strain ATCC 200989 and tested for geneticin resistance under uninduced and induced conditions (FIG. 3C). We found that this strain was relatively resistant to geneticin, requiring about 1 mg/ml geneticin to suppress growth with an empty pFPG1 plasmid. In the presence of 1 mg/ml geneticin, strain ATCC 200989 carrying the empty plasmid failed to grow under either uninduced or induced conditions, whereas the same strain carrying the pFPG1-NEO plasmid grew moderately well under uninduced conditions and well under induced conditions. This indicated that neomycin/kanamycin resistance was expressed from pFPG1. In this case, induction of the MT-1 promoter in pFPG1 was not absolutely necessary to obtain adequate expression of the NEO gene.

The second set of experiments were performed in a similar fashion with pFPG2, the version of the vector which carries the C. glabrata CEN-ARS region. One would expect this vector to be stably maintained in a lower copy number than pFPG1. Strain FSGO15 (C. glabrata Δade2) carrying the ADE2 gene on pFPG2 failed to grow under either uninduced or induced conditions, indicating that the level of expression from the pFPG2 plasmid is not enough to complement the ADE2 deletion. By contrast, strain FSGO20 (C. glabrata Aleu2) carrying the LEU2 gene on pFPG2 grew moderately well under induced conditions and failed to grow under uninduced conditions. This indicated that LEU2 expressed from pFPG2 was sufficient to complement the LEU2 deletion and that induction of the MT-1 promoter was necessary for adequate expression.

The NEO pFPG2 plasmid and empty pFPG2 as a control were transformed into C. glabrata strain ATCC 200989 and tested for geneticin resistance on -Ura medium under uninduced and induced conditions (FIG. 6). We found that this strain was relatively resistant to geneticin, requiring about 1 mg/ml geneticin to suppress growht with an empty pFPG2 plasmid. In the presence of 1 mg/ml geneticin, strain ATCC 200989 carrying the empty plasmid failed to grow under either uninduced or induced conditions, whereas the same strain carrying the NEO plasmid failed to grow under uninduced conditions and grew well under induced conditions. This indicated that neomycin/kanamycin resistance was expressed from pFPG2 and that expression of the gene required induction of the MT-1 promoter with copper. In summary, pFPG2 seems to exhibit lower expression levels which allowed regulated expression for the LEU2 and NEO genes.

We claim:

1. A vector for expressing a gene of interest in a Candida species, wherein said vector comprises:
a multiple cloning site;
promoter and terminator sequences from a native Candida gene flanking said multiple cloning site, wherein said gene is a regulated gene;
an autonomously replicating sequence from Candida; and
a centromere sequence from Candida.

2. The vector of claim 1, wherein said vector also comprises a gene of interest cloned into said multiple cloning site.

3. The vector of claim 1, wherein said vector further comprises a selectable marker.

4. The vector of claim 2, wherein selectable marker is selected from the group consisting of URA3, HIS3, LEU2, and TRP1.

5. The vector of claim 1, wherein said vector further comprises an E. coli antibiotic resistance gene.

6. The vector of claim 4, wherein said E. coli antibiotic resistance gene is amp.

7. The vector of claim 1, wherein said promoter and terminator sequences are derived from a Candida glabrata gene.

8. The vector of claim 7, wherein said gene is Candida glabrata metallothionein I (MT-1).

9. The vector of claim 1, wherein said centromere sequence is derived from the C. glabrata CEN sequence.

10. The vector of claim 1, wherein said Candida species is Candida glabrata.

11. The vector of claim 2, wherein said gene of interest is a gene from Candida glabrata.

12. The vector of claim 2, wherein said gene is an essential gene.

13. A method for complementing deleted genes in a Candida species comprising:
A) deleting a gene of interest from said Candida species; and
B) transforming said Candida species with the vector of claim 2, wherein said vector comprises the gene of interest deleted from said Candida species.

14. The method of claim 13, wherein said gene of interest is deleted from said Candida species using a method which further comprises:
A) constructing a deletion fragment which comprises:
(i) a left flank comprising at least 200 bp of sequence from the region upstream of the gene of interest;
(ii) a right flank comprising at least 200 bp of sequence from the region downstream of the gene of interest; and
(iii) a selectable marker to replace the gene of interest;
B) transforming the Candida species with the deletion fragment, selecting for the replacement marker, and verifying that the appropriate gene replacement was made.

15. The method of claim 14, wherein said deletion fragment is prepared by crossover PCR of SOEing.

16. A method for identifying genes essential for the growth of a Candida species, said method comprising:
A) deleting a gene from the genome of said Candida species;
B) studying the growth of the Candida species which has had a gene deleted and assaying growth inhibition; and
C) complementing the deleted gene using the vector of claim 21 to express the deleted gene and determining if expression of the deleted gene restores said Candida species growth.

17. The method of claim 16, wherein said Candida species is Candida glabrata.

18. A method for determining a function of a gene from a Candida species, said method comprising:
A) deleting a gene from the genome of a Candida species;
B) studying the effect deleting the gene from said Candida species has on its growth; and
C) complementing the deleted gene using the vector of claim 2 to express the deleted gene; and
D) determining if expression of the deleted gene restores the function lost when the gene was deleted.

19. The method of claim 18, wherein said Candida species is Candida glabrata.

20. A method for screening for novel anti-fungal compounds, said method comprising:
A) transforming a Candida species with the vector of claim 2 in order to overexpress the gene of interest in said species;
B) administering to said Candida species, and to a Candida species which has normal expression of said gene of interest, a compound not previously known to be anti-fungal; and
C) determining if said compound is an anti-fungal compound by comparing the amount of resistance said Candida species which overexpresses said gene of interest has to said compound to the amount of resistance said Candida species which has normal expression of said gene of interest has to said compound.

21. The method of claim 20, wherein said Candida species is Candida glabrata.

22. A method of treating a Candida infection in a mammal, said method comprising administering to said mammal a therapeutically effective amount of a anti-fungal compound determined to be effective by the method of claim 20.

23. A method of inhibiting the growth of Candida in vivo or in vitro, said method comprising contacting said Candida with a therapeutically effective amount of a anti-fungal compound determined to be effective by the method of claim 20.

24. A method for preparing an antifungal compound, comprising the steps of:
A) screening for novel anti-fungal compounds using a method comprising:
(i) transforming a Candida species with the vector of claim 2 in order to overexpress the gene of interest in said species;
(ii) administering to said Candida species, and to a Candida species which has normal expression of said gene of interest, a compound not previously known to be anti-fungal; and
(iii) determining if said compound is an anti-fungal compound by comparing the amount of resistance said Candida species which overexpresses said gene of interest has to said compound to the amount of resistance said Candida species which has wild-type expression of said gene of interest has to said compound; and
B) synthesizing said compound in an amount sufficient to provide said compound in a therapeutically effective amount to a patient.

25. The method of claim 24, wherein said Candida species is Candida glabrata.
